(19) United States
(12) Patent Application Publication
(10) Pub. No.: US 2004/0006282 A1
(43) Pub. Date: Jan. 8, 2004

(54) Methods and apparatus for using vaginal electrodes for anticipating and detecting optimal breeding time

(76) Inventor: Vaclav Kirsner, (US)

Correspondence Address: VACLAV KIRSNER, 83 DAVIS RANCH ROAD, BELLVUE, CO, 80512, US

(21) Appl. No.: 10/610,114

(22) Filed: Jun. 30, 2003

Related U.S. Application Data
(60) Provisional application No. 60/392,555, filed on Jul. 1, 2002.

(51) Int. Cl. A61B 10/00

(52) U.S. Cl. 600/551

(57) ABSTRACT

Two electrodes for contacting consistently a selected position in the vaginal fornix of a mammalian female such as a cow are brought to the desired position so as to allow daily measurements indicative of the follicular waves and then of the optimal time for insemination. The detection of the follicular waves makes it possible to anticipate optimal breeding time first by 10 days and then by 3 days. The monitoring of the animal's reproductive cycle consists of daily measurements of admittance or small AC current generated by time-varying voltage of small amplitude and sufficiently high frequency. The consistent electrode placement is achieved using a configuration capable of entering the fornix and/or engaging the cervical protrusion in one of two ways. In one type of embodiment the electrodes are imbedded at the distal end of a probe shaft that is attached at its proximal end to an electrode-orientation mark carrier such as a self-contained electronic assembly. In another type of embodiment the electrodes are imbedded on the surface of a vaginal insert that remains inserted permanently to maintain a consistent electrode position. The insert carries the electronic circuitry for the periodic measurements and for their telemetric transmission to a receiver on the outside of the animal's body.

### BACKGROUND OF THE INVENTION

1. Field of the Invention

The invention of this application relates to the breeding management of agricultural animals such as cows and more specifically to methods and apparatus for monitoring the animals' reproductive status so as to anticipate and then determine the optimal time for insemination.

2. Background and Prior Art

The business management techniques employed by the American dairy farmers and beef ranchers are advanced but the two industries incur substantial losses due to inefficient breeding. This is known to be caused by the unresolved problem of ineffective determination of the optimal timing of insemination, whether natural or artificial. The resulting lost time between consecutive calves is of great economic importance to the beef producers. In the dairy business, where only mother cows can produce milk, the cows should be made pregnant on a strict schedule in order to maximize milk production and profitability. This goal of strictly scheduled re-breeding is mostly not achieved, due to the absence of an efficient diagnostic method for the determination of the optimal breeding time.

The difficulties that the herdsmen face stem from the prevalent empirical approach to the determination of the breeding time, based on observations of the animals' behavior. The difficulties will be understood from the following brief review of the variability and the uncertainties in the physiological timing relationships involved in breeding management. The estrous or fertile period in cattle occurs approximately every 21 days (plus or minus 3 or 4 days). Estrus or heat lasts for a relatively short period of some 12-18 hours, with some authors setting the upper limit at 24 hours or longer and the lower limit at just a few hours. The width of the range of estrus duration may be both inherent as well as caused by the difficulties of heat detection. However, the fact is that heat is short and often occurs during the night hours. Whatever its duration, ovulation occurs a few hours later (about 6-12 hours, and perhaps later still, as there is again an uncertainty about its timing). With these variabilities, the time of insemination is generally selected empirically, based on experience and the individual business management practices.

If a cow is successfully inseminated, which appears to require insemination during the latter part of heat or within a few hours afterwards, she will be pregnant for approximately 280 days. Dairy cows are managed intensely and often bred exclusively by artificial insemination, with a view to making them pregnant again 45 to 60 days after calving. If the planned re-breeding estrus is not detected, the operator starts losing money. The cows that remain “open” past the goal date are estimated to cause a loss of about $3.00 per cow per day, and they obviously cannot be inseminated for another 21 days or so.

A number of methods for detecting estrus in cows have been employed, including for example rectal palpation of the cow's reproductive tract, which is not applicable on a large scale. A widely used method is observing the behavioral indications of estrus in individual cows. Many dairymen do this at the time the cows gather at the milking location to be milked, mostly in 12-hour intervals. The behavioral indications include increased physical activity, bowling, and mounting other cows as well as allowing herself to be mounted (the so-called standing heat or behavioral heat). Visual observation of cows' behavior has been the primary technique of estrus detection, even though only about 50% of cows in heat are detected by visual monitoring and even though up to about 30% of thus detected and selected cows are not actually in heat.

Several techniques have been employed to aid the detection of behavioral heat such as the use of chalking or painting the backsides of the cows being monitored, whereby the marking is rubbed off when the cow is mounted. Such marking approaches are subject to errors, for example from spurious mounting of cows that are not in heat. Numerous patents have been granted to inventors attempting improvements whereby the visual observation of visible marks was replaced by electronic registration of the mounting events. This type of invention is exemplified by the following two U.S. patents that may be considered as representative of this field.

U.S. Pat. No. 5,111,799 (Senger et al., May 12, 1992) describes a pressure detector for surgical implantation under the hide on the cow's back near the tail-head. His implant analyzes the duration of the mounting pressure (>5 seconds) and the number of mountings over a sliding 8 hour period. It indicates estrus when a threshold of at least 3 mounting events has been reached, by transmitting a telemetric signal to a receiver. Standard commercially available components are utilized.

The other representative patent in the field of radiotelemetered measures of cows' mounting activity is U.S. Pat. No. 5,542,431 (Starzl et al., Aug. 6, 1996). This patent describes a system centered on a pressure detector that is attached to the cow's backside on the outside of the body, either by a harness or by an adhesive. The system is highly complex in terms of both hardware and software, and the software utilizes a newly introduced concept of peak estrus value, claimed to be useful in determining an optimal, or at least desirable, breeding time. The concept of peak estrus assumes a substantially symmetrical distribution of mounting behavior, with peak estrus centrally located at the time of peak mounting activity, which is postulated to occur when the estrus hormones (estrogen, E2 and luteinizing hormone, LH) are expressed at their highest levels. As in the Senger patent, the onset of estrus is detected when a threshold mounting activity is reached. However, in this patent, the time span for determining the threshold number of mounting events is substantially shorter, about four hours as compared to Senger's eight hours, and the peak estrus time is then found by analysis of the further mounting activity that follows the onset of estrus.

In the marketplace, the commercially available system of Starzl et al. has had problems due to its high capital cost, due to the fact that the detector of the mounting events tends to be torn off the animals' backsides, and because the system may not be cost effective. It must be kept in mind that considerable numbers of cows exhibit no signs of heat even though they are fertile. Using behavioral heat as the indicator of optimal breeding time has this inherent flaw. This is the problem of the so-called silent heat in which estrogen in the blood is too low to induce behavioral estrus but high enough to stimulate luteinizing hormone release and subsequent ovulation. This is, of course, in addition to the variability and the fundamental uncertainties in the timing of the estrus and ovulation relationships, reviewed above. The mounting detectors monitor heat-induced behavior but not the internal physiological events associated with ovulation.

For these reasons there have been efforts to determine the optimal breeding time independently of the behavioral characteristics. Two approaches to the diagnostic problem belong here. One is the application of milk hormone assays that were developed from the laboratory procedures analyzing the blood concentration profiles of the hormones that control the reproductive cycle. In particular, progesterone assay kits have been made commercially available, allowing the livestock breeder to determine daily if the progesterone levels decreased as it happens during several days around estrus, and if they increased afterward as it happens after ovulation. These progesterone assays have not become the industry standard for breeding-time determination, and when they are used, the purpose is pregnancy detection and the diagnosis of non-pregnancy.

The other approach to estrus detection independent of the animal's behavior has been through the vaginal measurement of the electrical resistance of vaginal fluids, that is of the vaginal and/or cervical mucus secreted by the respective epithelia. The field utilizing this approach to agricultural breeding management opened up in Europe in the sixties and early seventies, with publications such as L. B. Aizinbudas and P. P. Doviltis, Zhivotnovodstvo 11, 68 (1962) “Electrometric method for specifying the time for inseminating cows”; ibid. idem., 28, 84 (1966) “Some results from testing an electrometric method of determining insemination times for cows”; Z. Veznik et al., Vet. Med. (Praha) 16, 163 (1971) “Progress in diagnostic methods in reproduction of farm animals”; D. Schams and H. D. Butz, Zuchthygiene 7, 49 (1972) “Relationship in time between estrus symptoms, variations in the electric resistance of the vaginal mucus, pre-ovulatory secretion of luteinizing hormone and ovulation in bovines”; and E. Metzger et al., ibid., 7, 56 (1972) “An instrument for testing the vaginal mucus for electric conductivity in the determination of estrus in bovines”. All the devices in this category are elongated cylindrical probes for insertion into the vagina, carrying at least two electrodes by definition, so as to satisfy the closed circuit requirement of the electrical measurement. Electrodes are the active elements made of an electrically conductive material and imbedded in the surface of the electrically insulating body of the probe:

U.S. Pat. No. 3,844,276 (McDougall, Oct. 29, 1974) claimed a mucus resistance-measuring probe with two electrodes, one of which was cylindrical in shape and at least 8 inches long, with the other electrode being a small disc at the bottom of a concave depression at the insertion end of the probe's body. Upon insertion, the concave depression at the insertion end collected mucus from the cervix and then the probe was withdrawn slightly. In this manner, only mucus and not the epithelial tissue was in contact with the small disc electrode during the measurements. On the other hand, the large cylindrical electrode ensured contact with the epithelial lining of the vagina, this lining usually having a mucous coating. Mucus resistance was measured with a standard ohmmeter. Resistance readings were around 400-500 ohms before and after estrus. The best time for insemination was indicated as the 24-hour period after the minimum resistance value of around 300 ohms was obtained.

U.S. Pat. No. 4,224,949 (Scott et al., Sep. 30, 1980) claimed the use of at least one pair of axially aligned electrodes for the measurement of bovine vaginal mucus with a standard ohmmeter. The ohmmeter provided AC current having a frequency between 1 kHz and 1 MHz (preferably 5-100 kHz) at a voltage between 1 Volt and 10 Volts peak-to-peak (preferred voltage was 3 Vpp-6 Vpp). The electrodes were about 1 to about 4 inches long (preferably about 2 to about 3½ inch long) and about 1/16 to about ½ inch wide. They were substantially parallel and aligned parallel to, or at an angle of less than 45 degrees from, the longitudinal axis of the probe body. As with the McDougall apparatus discussed above, the measurement procedure involved bringing the tip to the cervix first but then pulling back so that during measurements the electrodes were more than an inch away from the cervix and the fornix. Resistance of around 50 ohms was registered before and after estrus. Estrus was indicated by a drop in the resistance readings to a minimum of around 30-40 ohms. Estrus was more accurately detected as the mean of two measurements with two pairs of electrodes. In this configuration, the four parallel electrodes were placed all around the cylindrical surface of the probe, spaced 90 degrees apart. Ring shaped electrodes and spiral helix shaped electrodes were argued to be less sensitive for the mucus resistance measurement, and therefore undesirable electrode configurations.

U.S. Pat. No. 4,498,481 (Lemke, Feb. 12, 1985) claims the use of two spirally wound helix electrodes (gold- or platinum-plated) for the detection of a minimum resistance of mucus. This is declared to determine the appropriate time to breed the animal. Rather than the use of a standard ohmmeter, this patent discloses the use of a 25 kHz constant AC current generator for the resistance measurement and display, and claims this to be incorporated in a housing at the top of a flat upper surface of a pistol-grip handle. The patent discloses that, with this system, each animal has its own individual set of resistance reading levels, which can range from near zero to over 1000 ohms. As was the case with the other disclosures, the electrical resistance measurement indicates estrus as a minimum in the resistance readings. However, here the resistance measurement does not appear to be considered sufficient for diagnosis because it is complemented with visual observation of the cervix (via a fiber-optic scope) and with a temperature measurement.

Despite the desirability of an objective assessment of the optimal breeding time that would be independent of the animals' behavior, none of the vaginal probing techniques have achieved the desired success as tools for breeding management. One of the problems of the prior art is that individual animals have their individual ranges of measured mucus resistance values. Another problem is the inability to anticipate well in advance the approach of the optimal insemination time so as to be able to plan for it. The problem is due to the featureless, flat, appearance of the cyclic pattern of the electrical resistance readings before the single inflection at the time of estrus.

The same otherwise featureless curves with a single inflection (a minimum) about a day before ovulation have been disclosed as obtaining when the vaginal/cervical mucus resistance measurement was applied to the monitoring of human fertility status. U.S. Pat. No. 4,836,216 (Fernando et al. Jun. 6, 1989) in FIG. 4 and U.S. Pat. No. 5,240,010 (Weinmann, Aug. 31, 1993) in FIG. 3 show these single-feature vaginal cyclic profiles obtained in human females.

In contrast, I disclosed in my U.S. Pat. No. 4,753,247 (Kirsner, Jun. 28, 1988) a method of vaginal monitoring that does yield a human cyclic profile with a number of features facilitating a timely forecast of ovulation. Following additional research after the issuance of the patent, the method may now be referred to as the localized (capacitive) admittance method. In accordance with this method, admittance or AC current response to the application of a small-amplitude high-frequency voltage is monitored daily in the posterior fornix of the human vagina. The graph of the daily measurements in the human vagina includes a first peak, which provides a long-term forecast of the forthcoming ovulation. Then, a few days later, the number of which depends on the actual physiological conditions in the present reproductive cycle, a second peak gives a short-term warning of ovulation; subsequently, an ovulation marker is obtained. Such resolution of the reproductive cycle is not obtained with any of the methods and apparatuses of the prior art reviewed above. However, when applied to experimental agricultural animals including cows, a rather large scatter on the bovine cyclic profile data was observed, which would adversely affect the reliability, and therefore the usefulness, of the technique in commercial practice.

The reviewed prior art disclosures involve several characteristic features that cause their insufficient sensitivity in monitoring the animals' fertility status. The reviewed vaginal probing disclosures contain no teaching of having the electrodes located at the tip of the vaginal sensor body for a tissue contact in the vaginal fornix region; they do not teach any particular accommodation of key anatomical features. Their description of the respective probes involves no means for predetermined or consistent positioning and/or orientation of the electrodes in the vagina. The methods of the prior art patents do not use any sensor-orientation means and procedures. The prior art methods of other inventors generally avoid electrode contact with the tissues in the vaginal fornix region during the measurement. They aim instead to measure the resistance of the secreted mucous fluids, for which reason their methods involve wetting of the electrodes with the mucus. Invariably, the step of wetting the electrodes with the mucous fluids is followed by withdrawal of the probe from the vicinity of the cervix just before the measurement. In addition, the devices of the bovine vaginal probing prior art rely on electrode stimulation with very high electric currents, which is a detrimental feature from both the biological and economic viewpoints.

### SUMMARY OF THE INVENTION

The present patent application aims to resolve the problems of the prior art apparatus and methods. In accordance with the invention of the present application, a probe and a permanent vaginal insert are provided, along with various methods, which achieve consistent placement of attached electrodes for monitoring the fertility and pregnancy status of an animal by measurements in the vaginal fornix of the animal.

One embodiment of the invention is a probe for monitoring the reproductive status of an animal. The probe has an elongated body having a first end and a second end. The first end has a tip for engaging the cervical protrusion of the animal and for contacting tissues in the fornix region. The tip has at least two electrodes. The second end of the elongated body has marker means for permitting rotation of the elongated body to a predetermined radial orientation.

The electrodes of the probe can be oriented in a variety of ways. At least one of the electrodes can be placed at the end of the probe tip. At least one of the electrodes can be placed at the inner surface of the probe tip. At least one of the electrodes can be placed at the outer surface of the probe tip.

The tip of the probe also can be configured in a variety of ways. The tip can comprise a ring having an internal cavity for receipt of the cervical protrusion. Or the tip can taper into a blunt cone split at the apex and having an internal cavity for receipt of the cervical protrusion.

The marker means of the probe can be configured to permit the elongated body to be repeatedly rotated to the same radial orientation. The marker means can comprise a planar section of thin, rigid material that bears at least one orientation marking and is situated between the probe and a separate handle for the probe. The marker means can comprise a handle attached to the probe and a case attached to the handle. The case encloses apparatus for measuring the reproductive status of the animal, display means for indication of measurement results, and keyboard means with at least two groups of keys, one for controlling logic and keyboard functions and the other for entering data. The case is capable of rotating the probe to a predetermined radial orientation.

Another embodiment of the invention is a method for monitoring the reproductive status of an animal. In this method a probe is inserted into the vagina of the animal. The probe has at least two electrodes at its insertion end, and the insertion end is configured to fit the cervical protrusion of the animal. The probe is rotated to a predetermined radial orientation, an electrical signal is applied across the electrodes, and a signal indicative of the reproductive status of the animal is measured across the electrodes.

The insertion end of the probe can be pressed into contact with tissues of the fornix region after insertion of the probe. The probe can be inserted repeatedly into the vagina of the animal. The rotation of the probe can be gauged by marker means at the end of the probe opposite the insertion end of the probe.

Another embodiment of the invention is a probe for monitoring the reproductive status of an animal. The probe comprises an elongated body having a first end and a second end. The first end has a tip for entering the fornix region and contacting the tissues in that region. The tip has at least two electrodes spaced from the end of the tip such that the distance between the end of the tip and the proximal end of at least one of the electrodes does not exceed about 100% to 150% of the length of the cervical protrusion of the animal. The second end of the probe has a marker for gauging the rotation of the elongated body to a predetermined radial orientation.

Another embodiment of the invention is a method for monitoring the reproductive status of an animal. In this method, a probe is inserted into the vagina of the animal. The probe is equipped with a tip having at least two electrodes spaced from the end of the tip such that the distance between the end of the tip and the proximal end of at least one of the electrodes does not exceed about 100% to 150% of the length of the cervical protrusion of the animal. The probe also has an orientation marker at the end of the probe opposite to the tip-bearing end of the probe. The marker is rotated so as to rotate the probe to a predetermined radial orientation, an electrical signal is applied across the electrodes, and a signal indicative of the reproductive status of the animal is measured across the electrodes.

The tip can be pressed into contact with tissues of the fornix region after insertion of the probe. The probe can be inserted repeatedly into the vagina of the animal. The end of the probe opposite the tip can have marker means for rotation of the probe to a predetermined radial orientation.

Another embodiment of the invention is a vaginal insert for monitoring the reproductive status of an animal. The vaginal insert comprises a tubular body for insertion into the animal for extended periods, the body being configured to fit around the cervical protrusion of the animal, at least two electrodes fixed to the body, and a telemetry unit capable of imposing an electrical signal across the electrodes, measuring the output of the electrodes, and transmitting the measurement to a receiver.

The body can be a tubular torus. The body can be a spring wound into a cylindrical spiral that contracts during insertion into the animal and expands once inside the animal. The body can be a spring wound into a truncated conical spiral that contracts during insertion into the animal and expands once inside the animal.

At least one of the electrodes can be placed at the end of the body closest to the cervical protrusion. At least one of the electrodes can be placed at the inner surface of the body at the end of the body closest to the cervical protrusion. At least one of the electrodes can be placed at the outer surface of the body at the end of the body closest to the cervical protrusion.

Another embodiment of the invention is a method for monitoring the reproductive status of an animal. In this method, a vaginal insert having at least two electrodes and a cavity for receipt of the cervical protrusion of the animal is inserted into the vagina of the animal, an electrical signal is applied across the electrodes, a signal indicative of the reproductive status of the animal is measured across the electrodes, and the measurement is transmitted to a receiver.

Another embodiment of the invention is an insertable device for monitoring the reproductive status of an animal. The device comprises a body having a first end and a second end and means at the first end for engaging the cervical protrusion of the animal and for contacting tissues in the fornix region. The means has at least two electrodes. The device can have means at its second end for rotating the device to a predetermined radial orientation.

In accordance with the invention, it has been found that the localized admittance technique may be employed for the determination of the optimal breeding time in animals, even though the cyclic profile of the different mammalian species exhibits characteristics different from those in the human profile. Such species differences are not observed with the prior art methods of mucus resistance measurement. A number of means for consistent placement of electrodes, required for the localized admittance technique, are disclosed. The admittance technique consists of applying a low-amplitude, high-frequency voltage across two electrodes consistently positioned in contact with selected portion of the epithelia in the fornix of the vagina. The epithelial tissue-driven admittance readings, as a function of the day of cycle, track the several follicular waves of which only the last one yields the ovulating follicle. A long-term anticipation of the optimal breeding time is provided by a nadir between the first and second non-ovulating follicular waves. The ovulating follicular wave can be distinguished as a significantly higher wave of admittance readings with a crest that precedes ovulation. This allows the user to inseminate the animal at a selected phase of the cycle as defined by the admittance profile, such as on the day after the crest of the ovulating follicular wave.

The apparatus and methods according to the invention of this application provide a tool for animal breeding management that allows the breeder to arrive at the optimal timing of insemination by means of an objective physiological measure of the stage of the cycle. They are also useful for early pregnancy detection.

The invention of this application meets a number of objects.

One object of the invention is to improve the sensitivity, reliability and repeatability of prior methods and apparatus for vaginal monitoring of the fertility and pregnancy status in mammals in general and in bovines in particular.

Another object of the invention is to monitor a parameter characteristic of the physiological and/or endocrinological status of vaginal fornix tissues.

Another object of the invention is to enable the monitoring of follicular development.

Another object of the invention is to anticipate and determine estrus in animals independently of the behavioral expressions of estrus.

A final object of the invention is to provide a breeding management tool for dairymen, meat producers, veterinarians and/or other animal breeders.

### BRIEF DESCRIPTION OF THE DRAWINGS

FIG. 1 is a schematic illustration of the vaginal canal of a cow, showing the electrode contact with the epithelium in the fornix region according to the invention of the present application, in contrast to other electrode placements.

FIG. 2 depicts one example of prior art apparatus with indiscriminate vaginal tissue-electrode contacts.

FIG. 3 is another example of prior art apparatus with indiscriminate vaginal tissue-electrode contacts.

FIG. 4 is a schematic representation of the region of the vaginal fornix showing several radial orientations of the electrodes within the fornix.

FIG. 5 depicts the concept and one method of consistent radial orientation of the electrodes.

FIG. 6 is a schematic illustration of a vaginal probing apparatus for daily insertion according to the present invention.

FIG. 7 depicts one preferred embodiment of the probe tip in the shape of a blunt cone with several alternative placements of the electrode pair.

FIG. 8 shows another embodiment of the probe tip with the cone split open at the apex and with several alternative placements of the electrode pair.

FIG. 9 represents a probe tip embodiment in the form of a tapered ring attached to the end of a slim rod and with several alternative placements of the electrode pair.

FIG. 10 depicts one preferred embodiment of a permanent vaginal insert element in the shape of a tubular torus with several alternative placements of the electrode pair.

FIG. 11 shows another embodiment of the permanent vaginal insert element in the shape of a cylindrical spiral spring with wide turns and with several alternative placements of the electrode pair.

FIG. 12 depicts another preferred embodiment of the permanent vaginal insert element in the shape of a tapered helical spring or truncated conical spiral with wide turns and with several alternative placements of the electrode pair.

FIG. 13 is a graph of localized admittance measurements in a cow, showing two estrus occurrences 23 days apart and the follicular waves between them.

### DETAILED DESCRIPTION OF THE PREFERRED EMBODIMENTS

While the invention is susceptible to various modifications and alternative forms, specific embodiments are shown by way of example in the drawings and are described in detail below. The description and drawings are not intended to limit the invention to the particular embodiments disclosed. On the contrary, the invention embraces all modifications, equivalents, and alternatives falling within the spirit and scope of the invention as defined by the appended claims.

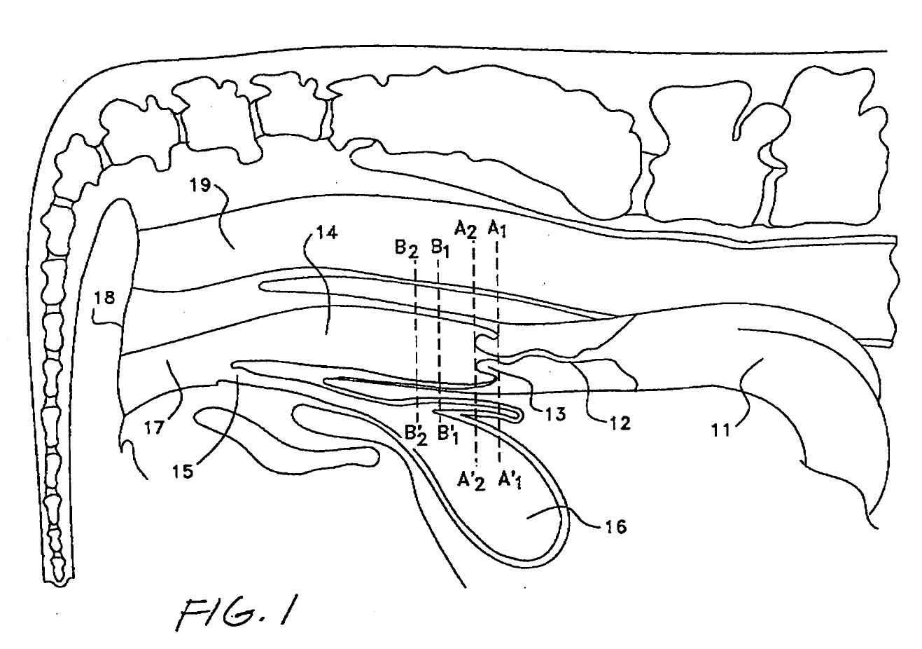

The present invention generally comprises apparatus and methods for achieving consistent electrode-tissue contact and thus consistent measurements of an electrical property, in particular of localized admittance, in the vaginal fornix of the cow or other animals. In order to help explain the invention, FIG. 1 depicts schematically the anatomy of the reproductive tract of the cow. Starting from the anterior aspect on the right of the figure, the uterus 11 ends in the cervix 12 shown with its centrally situated and somewhat curved cervical canal. The two-horned configuration of the bovine uterus is merely suggested on the right of the figure, and other details, that would depict uterine tubes leading to the ovaries, are not shown. The cervix 12 protrudes into the vagina and the cervical protrusion 13 is surrounded by the vaginal fornix. The fornix region is indicated on the schematic cross-section as the region limited by the line A1-A′1 representing the plane at the bottom of the cervical protrusion, and by the line A2-A′2 representing the plane at the tip of the cervical protrusion into the vagina. The distance between these two planes is the depth of the vaginal fornix. It is the target space for the placement of the electrodes according to the invention, as opposed to the other regions of the vaginal canal. Such undesirable regions of electrode contact are exemplified by the region depicted by the space between the planes represented by the lines B1-B′1 and B2-B′2. This space is situated away from the cervical protrusion 13. In the case of this example it is elsewhere within the anterior vagina 14. The anterior vagina part of the vaginal canal extends posteriorly to just before the urethral orifice 15 through which urine is expulsed from bladder 16. The urinary bladder 16 is connected at the urethral orifice 15 to the vaginal vestibule or posterior vagina 17. The vulva 18 separates the vaginal vestibule from the outside, and consists of two vertical lips that enclose a vertical slit-like opening. Rectum 19 runs substantially parallel above the vaginal canal.

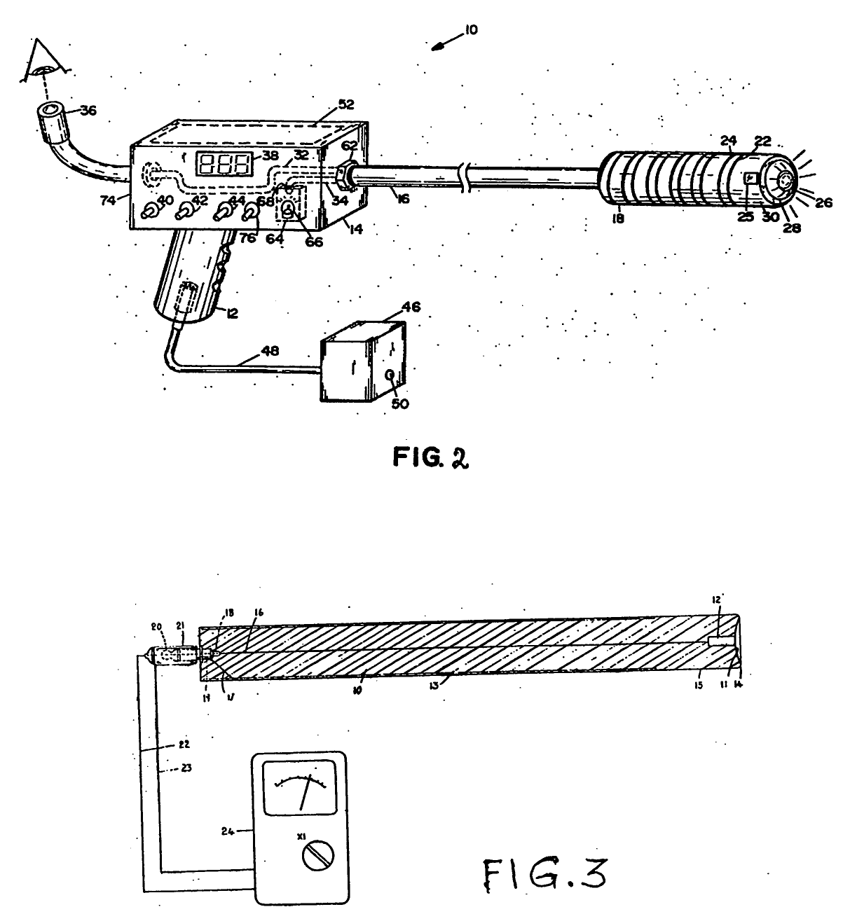

FIG. 2 illustrates the apparatus of the prior art that, unlike the present invention, did not aim at consistent electrode placement and was designed for mucus resistance measurements. The figure is from U.S. Pat. No. 4,498,481 (Lemke, Feb. 12, 1985) cited above in the Background and Prior Art section, and it shows a set of twin helix electrodes 22 and 24 imbedded in a plastic probe housing 18. The electrodes are wound along the whole length of the probe 18. The electrodes are placed too far away from the epithelia of the cervix and the fornix, and are placed indiscriminately along a large portion of the vaginal canal.

Another illustrative example of prior art apparatus is in FIG. 3, which is from U.S. Pat. No. 3,844,276 (McDougall, Oct. 29, 1974) that was also discussed in the Background and Prior Art section. The cylindrical electrode 13 extends over the major length of the probe so as to make contact with the vaginal fluids and the epithelial lining along substantially the whole of the vaginal canal. The other electrode 12 contacts only the mucus collected in the concavity 11, having been brought into momentary contact with the cervix so as to collect the mucus and then be pulled back. In this manner the contact with the cervix is avoided during the resistance measurement. The step of pulling back away from the cervix just before the measurement is typical of the prior art of other inventors including that of U.S. Pat. No. 4,224,949 (Scott et al., Sep. 30, 1980) discussed in the Background and Prior Art section above. This procedural step of withdrawing from the cervix is consistent with their designs to measure the resistance of the mucous fluids, rather than measuring an electrical property of a targeted epithelium as is the object of the present disclosure.

FIG. 4 is a schematic representation of the fornix region, which is the space surrounding the cervical protrusion and bounded on the outside by the epithelium of the vaginal wall 41. The figure is a simplified cut out perspective view, depicting a number of possible orientations of the placement of an electrode pair when introduced into the fornix region. The cervical protrusion is schematically represented as the simple sideways-oriented hill-like structure 42, with the opening of the cervical canal 43 at its apex. The bottom of the fornix is pointed out at two points 44 and 45 that are 180 degrees apart while the fornix surrounds the cervical protrusion 42 all around, and its bottom extends over all of the 360 degrees. The depth of the fornix region was explained in FIG. 1 as the distance between the bottom and the tip of the cervical protrusion. If unguided, a pair of electrodes, comparable in size to the depth of the fornix region and spaced 180 degrees apart, could be placed into the fornix region so as to be oriented at any angle. Four of the multitude of orientations are depicted by lines connecting the pairs of points A-A′, B-B′, C-C′, and D-D′. The placement of the electrodes according to this invention should be consistent throughout the period of monitoring of the animal's reproductive cycle. Tissues targeted for electrode contact are the segment of the epithelium of the vaginal wall 41 that is within the depth of the fornix region, and/or the epithelium of the cervical protrusion 42, and/or the bottom of the fornix (e.g., 44, 45). The sideways hill-like representation of the bovine cervical protrusion in FIG. 4 is an oversimplification as in reality the protrusion is not so conical but rather irregular in shape.

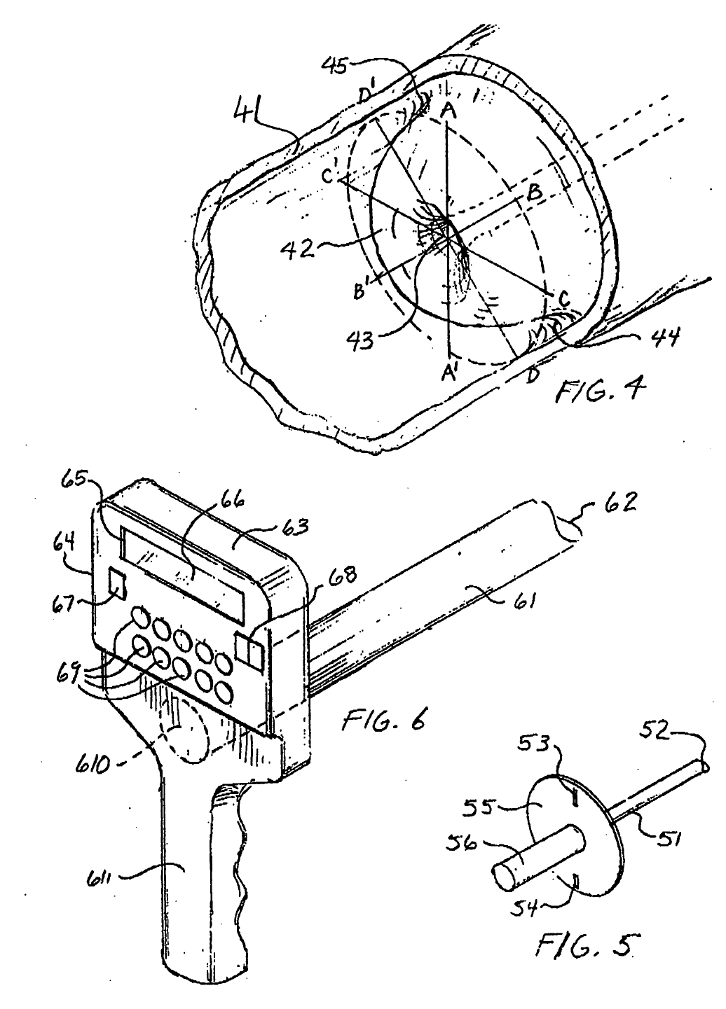

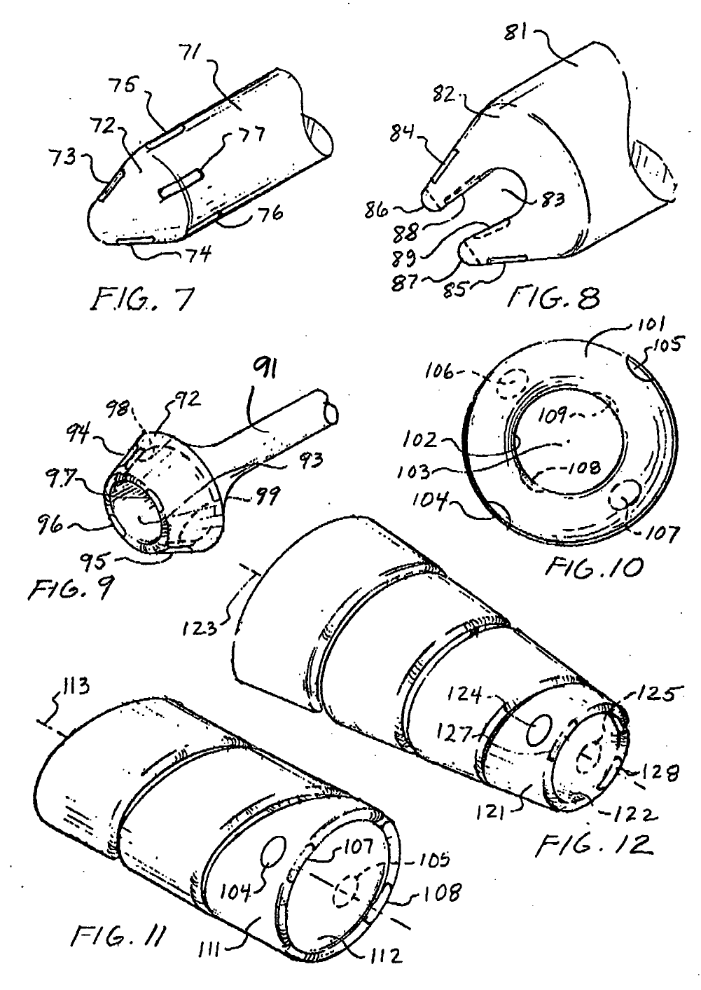

FIG. 5 illustrates the concept and one method of consistent placement of the electrodes in the selected orientation. The figure pertains to a method and apparatus for introducing the electrodes periodically by means of a probe that is elongated and cylindrical in shape. The figure depicts the proximal end of the probe, which is the end that is held by and is visible to the user during the process of inserting the probe and during the measurement. The arrangement of the electrodes, mounted at the tip of the insertion end of the probe body 51 further beyond the point 52, will be described in FIGS. 7-9. Unlike in the prior art probes, the electrodes are mounted in such a manner as to be aligned with, or oriented with respect to, the marks 53 and 54 that appear on a circular or otherwise suitably shaped planar piece 55 at the distal end of handle 56. In this manner, introducing the probe consistently with marks 53 and 54 in the same orientation, the electrodes at the insertion end are correspondingly always placed in the same selected radial orientation. As an alternative or a complement to the illustrated marks 53 and 54, the marker means for consistent radial orientation of the electrodes may involve a level indicator, including but not limited to a sight level or an electronic level indicator. The connection of the probe electrodes to the electronic part of the apparatus is not shown in FIG. 5; the electronic part may be contained in a separate box connected by cable to the probe or it may be incorporated in a single-piece arrangement as shown in FIG. 6.

An example of a suitable electrode stimulation for bovine reproductive status measurements by means of the localized admittance technique is inter-electrode voltage of 100 millivolts peak to peak at the frequency of 50 kHz. The amplitude of the voltage is up to hundred times lower than the voltages employed in the bovine prior art disclosures. The measured admittance currents are on the order of several hundred microamperes.

FIG. 6 shows one other method and apparatus for the consistent placement of the electrodes for bovine reproductive cycle monitoring. As was the case with the probe in FIG. 5, the probe body 61 has the electrodes mounted further beyond the point 62, as will be described in FIGS. 7-9. FIG. 6 illustrates a preferred embodiment of the invention in which the probe body 61 is attached directly to the case 63 containing the electronic components of the apparatus. The consistent orientation of the electrodes is secured by alignment with the vertical edge 64 of the case 63 of the apparatus, and/or with the vertical edge 65 of the display 66. Both of these edges are held in the vertical orientation during the process of insertion and particularly during the actual measurement. The marker means can alternatively consist of a level indicator such as a sight level or an electronic level indicator associated with the display 66. In this manner, having inserted the insertion end so as to reach the bottom of the fornix, the electrodes are consistently placed in the same position.

FIG. 6 also depicts some other features of this preferred embodiment, as follows. Under the display 66 is a keyboard with at least two groups of keys. One group is exemplified by keys 67 and 68 whereby key 67 is the on/off key and 68 is a confirm/delete rocker key or a pair of separate keys. The other group of keys 69 makes it possible to enter data such as an animal identification number. This preferred embodiment of the invention is provided with an electronic memory to hold data on a group of animals, using the animal identification numbers that distinguish the individual sets of data from each other. The data can be downloaded via a port that is not shown. The keyboard is physically located above the region 610 of the casing 63 where the probe body 61 is attached to the casing, on the side facing away from the user, which is the side that faces the animal's backside. The distance between the keyboard and the lower edge of the region 610 is such as to be sufficient to allow exerting some pressure on the probe during the process of insertion, without inadvertently hitting the keys in the process. For this reason, the lower part of the user face that contains the region 610 is recessed with respect to the upper part that holds the interface. Still lower below the region 610 is the handle part 611 of the apparatus. The probe-insertion pressure may be applied, for example, by the thumb of the hand that holds the handle part 611.

FIG. 7 shows one preferred embodiment of the probe tip with the electrode arrangement at the insertion end part of the apparatus. The insertion end is the end of the part of the probe body that extends well beyond point 52 in FIG. 5 and/or beyond point 62 in FIG. 6. The tip has the shape of a blunt cone 72 carried at the end of a cylindrical rod 71. Like the rest of the probe, the rod and the tip are made of an electrically insulating biocompatible plastic material. Also as with the rest of the probe, the dimensions of the insertion end are compatible with the animal that is to be monitored with the apparatus. For a cow, the rod 71 has approximately 20 to approximately 35 millimeters external diameter. The tip 72 carries on its surface two electrodes 73 and 74 of oblong or rectangular shape, approximately 10 to 15 mm long and 2 to 4 mm wide. Other electrode shapes are consistent with the invention, such as circular or square, as are suitable for achieving sufficient contact and level of the admittance signal.

The electrodes are imbedded flush with the surface of the cone and placed usually 180 degrees apart with respect to the axis of the rod 71. Other relative displacements and orientations are possible such as a displacement angle of less than 180 degrees, including zero degrees where one of the electrodes is closer to the apex of the tip than the other electrode. The separation distance of the insertion tip is approximately 300 to 500 millimeters from the orientation mark-carrying planar piece 55 in FIG. 5 or from the animal-facing side of the case 63 in FIG. 6. The insertion end 71 is carried at the end of a probe body shaft, which may be slimmer than the insertion end part itself. Probe body 51 in FIG. 5 and 61 in FIG. 6 are both respective parts of the said probe body shaft.

With a cone tip of a sufficiently small height (relative to the size of the cervical protrusion), the electrodes may alternatively be positioned just behind the cone and immediately adjacent to its base, as shown with electrodes 75 and 76. The electrodes are configured so that they make contact with the tissues in the fornix region and do not extend beyond. The proximal end of any of the electrodes, that is the end or edge farthest from the bottom of the fornix (44, 45 in FIG. 4), does not exceed about 100% to 150% of the length of the cervical protrusion. This specification excludes the prior art probe of Scott et al. (U.S. Pat. No. 4,224,949). Their electrode contact extended well beyond the fornix region because their electrodes were several inches long (claimed about 1 to about 4 inches, reportedly used 3 inches). Their electrodes were mounted about 1 inch away from the tip. Furthermore, in their measurement procedure, their probe was inserted first until the tip touched the tip of the cervical protrusion but then it was pulled away from the fornix region prior to the measurement, which caused electrode contact with a range of tissues other than in the fornix. This is contrary to the invention of the present application, as was represented in relation to the wrong contact space delineated schematically by lines B1-B′1 and B2-B′2 in FIG. 1.

The electrodes according to the present invention may also be imbedded so as to bend forward with the surface of the insertion end across the transition from the rod into the cone tip. This is illustrated with electrode 77 on the near side of the tip. For this case, the second electrode that may be symmetrically positioned on the far side of the tip is not shown in FIG. 7.

FIG. 8 is a representation of three other embodiments of the invention, with electrodes of similar dimensions and flush surface mounting as was the case in FIG. 7. The cylindrical rod of the insertion end 81 tapers into a blunt cone 82 that is split open at the apex to create an internal space 83. This space 83 is provided to receive the cervical protrusion. In one embodiment, electrodes 84 and 85 are mounted on the outside of the open cone and make contact with the tissues of the fornix on the vaginal wall surrounding the cervical protrusion. In another embodiment, electrodes 86 and 87 are mounted on the apex of the split cone and make contact with the epithelium at the bottom of the fornix. In another embodiment, the electrode pair 88 and 89 is mounted on the inside of the opening 83 and provides for contacting the cervix.

FIG. 9 represents a modification of the probe tip of FIG. 8 for the set of the three alternative electrode-mounting arrangements, with electrodes of similar characteristics as in FIG. 8. The rod 91 holds a tapered ring 92 with internal space 93 provided to receive the cervical protrusion. The ring wall therefore fills the space of the fornix region as it surrounds the cervical protrusion. In one embodiment, electrodes 94 and 95 are mounted on the outside of the ring wall. In another embodiment the electrodes 96 and 97 are mounted on the leading edge of the ring. In yet another embodiment the electrodes 98 and 99 are mounted on the internal surface of the ring wall.

As was the case with the tip depicted in FIG. 7, the dimensions of the tips shown in FIGS. 8 and 9 are compatible with the monitored animal and its reproductive tract. Not depicted in FIGS. 7-9 is the fact that the electrodes are connected by insulated wires to the electronic part of the probe. The electronic part is either attached directly to the probe body as shown in FIG. 6 or is contained in a separate box connected with the probe by cable (not shown).

The procedure used in the periodic measurement of the reproductive status of the animals involves entering the animal identification number via keys 69 and washing the probe with a disinfectant and water between animals, before inserting the probe into the vagina through the carefully cleaned vulva. The probe is inserted all the way to the bottom of the fornix, using the cervical protrusion as a guide for electrode positioning with respect to the cervical protrusion and using the marker means to secure the proper orientation of the electrodes within the fornix. In the case of any of the probe tip configurations represented in FIG. 7, the user should use the cervical protrusion to guide the probe so as to consistently place the tip in a selected position around the protrusion. For example, the tip may be directed always to the right as represented by point 44 in FIG. 4 (as opposed to the left side 45 or any other position). The tip configurations represented in FIGS. 8 and 9 are guided so as to receive the cervical protrusion, using the marker means for proper electrode orientation. The tip is pressed into the bottom of the fornix and the measurement is made while holding the probe firmly in its place for a few seconds. In one preferred embodiment, the readout will indicate the diagnostic meaning of the measured admittance and optionally provide a simple graph of the current cycle data, as well as storing the raw data in its memory, in the given animal's data file.

FIGS. 10-12 show several embodiments that are characterized by a wireless connection between the electrodes and the readout electronics, for long-term placement in the individual animals' vaginas. This type of embodiment is referred to as a vaginal insert. The insert can be manufactured from a suitable biocompatible plastic material that is non-irritating to the biological tissues. Examples are plastisol and the medical-grade Silastic polymers. The vaginal insert contains within its body the microelectronic circuit required to perform the measurement and transmit the result to an associated receiver part of the monitoring system on the outside of the animal's body. Different types of devices designed for long term use in animals' vaginas have been commercially available and so have telemetric systems for animal monitoring applications (e.g., from Wildlife Materials Inc. of Carbondale, Ill.). The vaginal insert can also be equipped to transmit a radial orientation information from an electronic level indicator useful particularly for the initial fitting of the vaginal insert.

FIG. 10 represents a vaginal insert in the shape of a tubular torus 101. This is fitted permanently into the fornix of the animal's vagina so as to hug the cervical protrusion with the inside wall 102, whereby the cervical protrusion comes through the hole 103 of the tubular torus body 101. In one embodiment, the electrodes 104 and 105 are mounted on the outer, larger-diameter, wall of the circular element to make contact with the tissues of the fornix on the vaginal wall surrounding the cervix. In another embodiment the electrodes 106 and 107 are mounted on the bottom-side wall of the torus element to make contact with the epithelium at the bottom of the fornix. In yet another embodiment the electrodes 108 and 109 are mounted on the inside wall of the element's opening 103 and provide for contacting the cervix.

FIG. 11 depicts a preferred embodiment of the vaginal insert in the form of a spiral spring with wide turns and with the electrode pair imbedded in one of three alternative positions at the front turn 111 of the spiral. The spiral spring is capable of contracting during insertion into the animal and is capable of expanding once inside the animal. Upon insertion, the cervical protrusion is surrounded by one or two turns of the spiral, which is fitted so that the front edge of the front turn 111 sits against the bottom of the fornix. The diameter of the spiral is consistent with the anatomical dimensions of the fornix region of the vagina. It is selected so that, when fitted, the spiral touches the cervical protrusion with the inner wall 112, and it presses gently outward along the whole length of the element. This distributes the mild pressure symmetrically away from the axis 113 along the vaginal wall, avoiding any irritation that might otherwise result from a localized pressure force. The length of the spiral exceeds significantly the length of the cervical protrusion into the vagina, at least by a factor of two or three. In one embodiment the electrodes 104 and 105 are mounted on the outer surface while in another embodiment they are mounted on the inner surface of the front turn of the spiral. In yet another embodiment the electrodes 107 and 108 are mounted on the front edge of the spiral for a tissue contact at the bottom of the fornix.

FIG. 12 represents a tapered modification of the spiral insert of FIG. 11 for the set of the three alternative electrode-mounting arrangements, with electrode pairs of similar characteristics as in FIG. 11. The taper of the truncated conical spiral is oriented with the wider end away from the front turn 121. The inner wall 122 of the front turn hugs the cervical protrusion while the mild outward pressure, which helps to hold the insert element in place, is achieved progressively more by the more distant turns of the spiral located away from the cervix. In one embodiment the electrodes 124 and 125 are imbedded on the outer surface while in another embodiment they are mounted on the inner surface 122 of the front turn of the spiral. In yet another embodiment the electrodes 127 and 128 are imbedded on the front edge of the front turn.

The method of using the vaginal insert for monitoring of the fertility or pregnancy status of an animal includes the important step of introducing the insert into the vaginal fornix at the beginning of the monitoring period, fitting it around the cervical protrusion. This fitting procedure may involve the use of an electronic level detector, incorporated into the microelectronic telemetric circuit within the body of the insert and transmitting the orientation information to an auxiliary indicator used by the person performing the fitting. The level indication, representing the orientation of the electrodes on the insert, may be a visual or an audio signal with suitable differentiation between the left and right tilts and the horizontal level position. Once fitted, the insert is under control of an electronic receiver on the outside of the animal body. Upon command, the telemetric circuit applies a signal across the electrodes, measures the response signal indicative of the fertility or pregnancy status, and transmits the response data to the receiver.

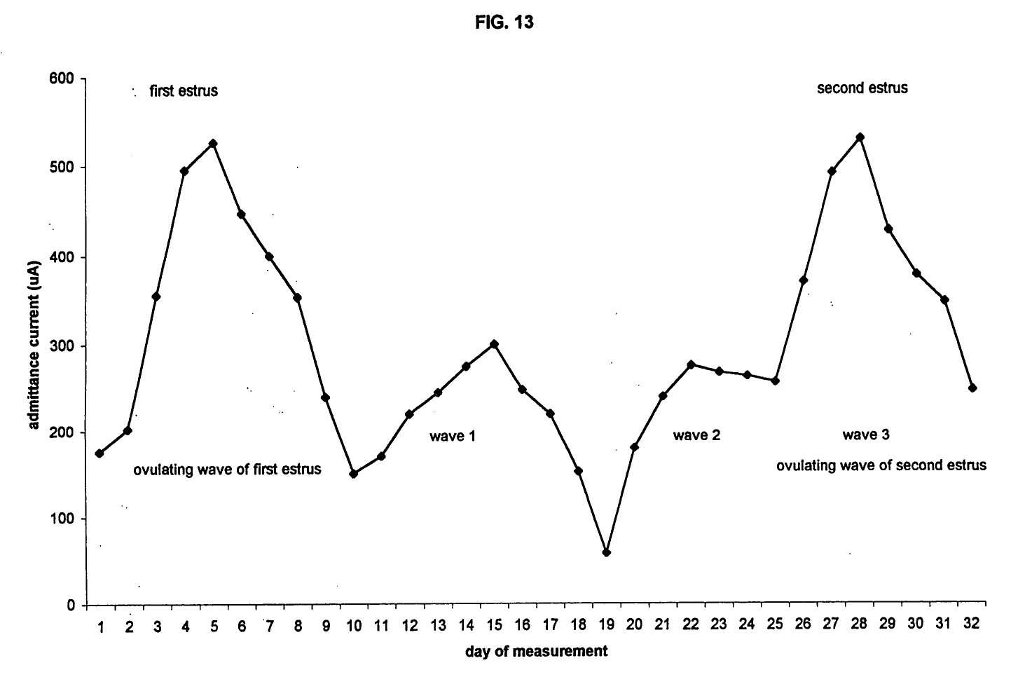

FIG. 13 illustrates the results of localized admittance measurements in a cow over a 32-days period. During the recorded period, two estruses are seen to have occurred as indicated by the two large ovulating follicular waves of the first and the second estrus, respectively. The signs of behavioral estrus will be observed on or about the day after the apex of the ovulating follicular wave. After the large ovulating wave of the first estrus, two small non-ovulating waves are seen in the graph. The graph shown in FIG. 13 indicates that the localized admittance detection of the ovulating follicular wave enables the anticipation of estrus by three days. In addition, the nadir that occurs between the first and the second follicular waves, after the first estrus, enables the anticipation of the next estrus by at least 10 days.

This technique of monitoring of the bovine fertility cycle provides a tool for breeding management by means of which the optimal timing of insemination can be established by livestock breeders. To improve their performance, the users of the technique will be able to correlate their breeding results with the timing of breeding procedures as tracked by the disclosed method and apparatus. The optimum time of insemination should be determined by correlating the obtained breeding results with inseminations performed at certain points along the ovulating follicular wave. The method is also useful for early pregnancy detection, which is something that behavioral estrus detection methods cannot provide. Pregnancy detection is provided by the absence of the follicular wave pattern including particularly the absence of the large ovulating follicular wave in the pregnant cow's record.

The described technique therefore provides a breeding management tool for the monitoring of the reproductive or estrous cycle as well as for the detection of pregnancy. These two separate but related functions, namely determining the fertility status or the pregnancy status, can be collectively referred to as the determination of the reproductive status. When the phrase “reproductive status” is used in this application, it encompasses all of the following: monitoring the fertility status that is the detection of the individual follicular waves during the estrous cycle, including the long-term predictive nadir and the large wave or peak before estrus and ovulation, as well as the distortion of the cyclic or follicular wave pattern by the state of pregnancy.

The embodiments discussed above are merely exemplary of the invention of this application. They are not intended to limit the scope of the claims of this application in any respect. Many devices and techniques different from those discussed above can be used in practicing the invention of the application. Only preferred embodiments and minor variants thereof have been shown and described above, and all changes and modifications that come within the spirit of the invention are intended to be protected.

What is claimed is:

1. A probe for monitoring the reproductive status of an animal, said probe comprising:
an elongated body having a first end and a second end,
said first end having a tip for engaging the cervical protrusion of the animal and for contacting tissues in the fornix region, said tip having at least two electrodes, and
said second end having marker means for permitting said elongated body to be rotated to a predetermined radial orientation.

2. The probe of claim 1 wherein said marker means permit said elongated body to be repeatedly rotated to the same radial orientation.

3. The probe of claim 1 wherein at least one of said electrodes is placed at the end of said tip.

4. The probe of claim 1 wherein at least one of said electrodes is placed at the inner surface of said tip.

5. The probe of claim 1 wherein at least one of said electrodes is placed at the outer surface of said tip.

6. The probe of claim 1 wherein said tip comprises a ring having an internal cavity for the receipt of said cervical protrusion.

7. The probe of claim 1 wherein said tip tapers into a blunt cone split at the apex and having an internal cavity for receipt of said cervical protrusion.

8. The probe of claim 1 wherein said marker means comprise:
a planar section of thin, rigid material, and
said section bearing at least one orientation marking and being situated between said probe and a separate handle for said probe.

9. The probe of claim 1 wherein said marker means comprise:
a handle attached to said probe, and
a case attached to said handle, said case enclosing:
apparatus for measuring the reproductive status of the animal,
display means for indication of measurement results,
keyboard means with at least two groups of keys, one of said groups for controlling logic and keyboard functions, and the other of said groups for entering data, and
said case being capable of rotating said probe to a predetermined radial orientation.

10. A method for monitoring the reproductive status of an animal, said method comprising the following steps:
inserting into the vagina of the animal a probe having at least two electrodes at its insertion end, said end being configured to fit the cervical protrusion of the animal,
rotating said probe to a predetermined radial orientation,
applying across said electrodes an electrical signal, and
measuring across said electrodes a signal indicative of the reproductive status of the animal.

11. The method of claim 10 wherein, in said insertion step, said insertion end is pressed into contact with tissues of the fornix region after insertion of said probe.

12. The method of claim 10 wherein, in said insertion step, said probe is inserted repeatedly into the vagina of the animal.

13. The method of claim 10 wherein, in said rotation step, said rotation is gauged by marker means at the end of said probe opposite the insertion end of said probe.

14. A probe for monitoring the reproductive status of an animal, said probe comprising:
an elongated body having a first end and a second end,
said first end having a tip for entering the fornix region and contacting the tissues in said region,
said tip having at least two electrodes spaced from the end of said tip such that the distance between the end of said tip and the proximal end of at least one of said electrodes does not exceed about 100% to 150% of the length of the cervical protrusion of the animal, and
said second end having a marker for gauging the rotation of said elongated body to a predetermined radial orientation.

15. A method for monitoring the reproductive status of an animal, said method comprising the following steps:
inserting into the vagina of the animal a probe equipped with a tip having at least two electrodes spaced from the end of said tip such that the distance between the end of said tip and the proximal end of at least one of said electrodes does not exceed about 100% to 150% of the length of the cervical protrusion of the animal, and further having an orientation marker at the end of said probe opposite to the tip-bearing end of said probe,
rotating said marker to rotate said probe to a predetermined radial orientation,
applying across said electrodes an electrical signal, and
measuring across said electrodes a signal indicative of the reproductive status of the animal.

16. The method of claim 15 wherein, in said insertion step, said tip is pressed into contact with tissues of the fornix region after insertion of said probe.

17. The method of claim 15 wherein, in said insertion step, said probe is inserted repeatedly into the vagina of the animal.

18. The method of claim 15 wherein, in said rotation step, the end of said probe opposite said tip has marker means for rotation of said probe to a predetermined radial orientation.

19. A vaginal insert for monitoring the reproductive status of an animal, said vaginal insert comprising:
a tubular body for insertion into the animal for extended periods, said body being configured to fit around the cervical protrusion of the animal,
at least two electrodes fixed to said body, and
a telemetry unit capable of imposing an electrical signal across said electrodes, measuring the output of said electrodes, and transmitting said measurement to a receiver.

20. The insert of claim 19 wherein at least one of said electrodes is placed at the end of said body closest to said cervical protrusion.

21. The insert of claim 19 wherein at least one of said electrodes is placed at the inner surface of said body at the end of said body closest to said cervical protrusion.

22. The insert of claim 19 wherein at least one of said electrodes is placed at the outer surface of said body at the end of said body closest to said cervical protrusion.

23. The insert of claim 19 wherein said body is a tubular torus.

24. The insert of claim 19 wherein said body is a spring wound into a cylindrical spiral that contracts during insertion into the animal and expands once inside the animal.

25. The insert of claim 19 wherein said body is a spring wound into a truncated conical spiral that contracts during insertion into the animal and expands once inside the animal.

26. A method for monitoring the reproductive status of an animal, said method comprising the following steps:
inserting into the vagina of the animal a vaginal insert having at least two electrodes and a cavity for receipt of the cervical protrusion of the animal,
applying across said electrodes an electrical signal,
measuring across said electrodes a signal indicative of the reproductive status of the animal, and
transmitting said measurement to a receiver.

27. An insertable device for monitoring the reproductive status of an animal, said device comprising;
a body having a first end and a second end, and
means at said first end for engaging the cervical protrusion of the animal and for contacting tissues in the fornix region, said means having at least two electrodes.

28. The device of claim 27 having means at said second end for rotating said device to a predetermined radial orientation.
